(19) United States
(12) Patent Application Publication
(10) Pub. No.: US 2022/0167931 A1
(43) Pub. Date: Jun. 2, 2022

(54) Wearable detection & treating device

(71) Applicants: Peter WEISZ, Shoham (IL); Assaf GRUNDMAN, Tel-Aviv (IL)

(72) Inventors: Peter WEISZ, Shoham (IL); Assaf GRUNDMAN, Tel-Aviv (IL)

(21) Appl. No.: 17/109,934

(22) Filed: Dec. 2, 2020

(51) Int. Cl. A61B 5/00; G16H 40/67; G16H 50/20; G16H 20/13; G16H 10/60; A61B 5/0205

(52) U.S. Cl. CPC A61B 5/746; A61B 5/1102; G16H 50/20; G16H 20/13; G16H 10/60; A61B 5/0022; A61B 5/6898; A61B 5/7267; A61B 5/6803; A61B 5/681; A61B 5/02055; A61B 5/4839; A61B 5/6843; A61B 5/7278; G16H 40/67

(57) ABSTRACT

The present invention provides a health monitoring system for alerting health condition of a user, said system comprised of:
At least on Replaceable elastic or semi elastic temple tip, design to be connected to a one end or part of a frame of existing eye glasses, said temple tip including at least one optic sensor, acceleration sensor or ballistocardiograph (BCG) sensor, CPU, memory, energy source and a communication module;
Monitoring application implemented on at least one computerized module, for aggregating raw measurement data, analyzing raw data using health oriented AI model for prediction of health conditions and providing alerts to the user and/or entity based on said prediction.

### FIELD OF THE INVENTION

The present invention relates to medical devices. More specifically, the present invention relates to a wearable detection device for monitoring multiple vital signs, cardiac biomarkers and for providing controllable treatment to the user.

### BACKGROUND OF THE INVENTION

Biosignal interfaces provide important data that reveal the physical status of a user, and they are used in the medical field for patient health status monitoring, medical automation, or rehabilitation services. Biosignals can be used in developing new contents, in conjunction with virtual reality, and are important factors for extracting vital signals of the user.

Biosignal interfaces provide important data that display the physical status of a user, and they are used not only in the medical field but also various other areas. In the medical field, they are used in monitoring systems for early detection of dangerous situations and diseases by monitoring the patient's health status and in medical automation systems that provide continuous treatment or rehabilitation services.

Thanks to this type of instrumentation, it is possible to continuously monitor patients and access their historical medical data. In this context, portable devices present great advantages since they allow this continuous monitoring without requiring the patient to remain hospitalized, unlike clinical instrumentation, which is not easy to transport and requires medical personnel for its configuration and manipulation.

### SUMMARY OF THE INVENTION

The present invention provides A health monitoring system for alerting health condition of a user, said system comprised of:
At least on Replaceable elastic or semi elastic temple tip, design to be connected to a one end or part of a frame of existing eyeglasses, said temple tip including at least one optic sensor, acceleration sensor or ballistocardiograph (BCG) sensor, CPU, memory, energy source and a communication module;
Monitoring application implemented on at least one computerized module, for aggregating raw measurement data, analyzing raw data using health-oriented AI model for prediction of health conditions and providing alerts to the user and/or entity based on said prediction.

According to some embodiments of the present invention, the health monitoring system wherein said optical sensor is a light reflecting sensor or a photodiode sensor or an ambient light sensor.

According to some embodiments of the present invention said elastic or semi elastic temple tip further comprises of at least one additional sensor from the list of: temperature sensors, humidity sensor, vibration sensor, audio sensor, wherein the AI model is training on integration of optical sensor data and the additional sensor data.

According to some embodiments of the present invention the monitoring module is implemented on a cloud server.

According to some embodiments of the present invention the monitoring module is implemented on smart phone device.

According to some embodiments of the present invention the computerized modules are partly implemented on cloud server and partly on the smart phone, wherein the smart phone module provides sensor data of the smart phone and user profile, enabling integrated analysis of temple tip sensor data and smart phone sensor data and profile data.

According to some embodiments of the present invention the systems further comprising calibration module for checking signal quality parameters for alerting user of adjusting position of the temple tip for improving signal quality. According to some embodiments of the present invention the Replaceable elastic temple tip is configured to be bent in toward and/or inward from the designated area of measurement, enabling to adjust in length, height and depth to provide both optimal measurements and optical usability of the eyewear frame, wherein the designated area of measurement is one of:behind ear, in front of the ear or inside the ear.

According to some embodiments of the present invention the replaceable elastic temple tip is configured to move backward of forward, enabling the user to adjust the temple tip position to achieve optimal measurement results by notifying the user if the temple tip needs to be adjusted.

According to some embodiments of the present invention the Replaceable elastic temple further includes a reservoir which includes a medical substance, connected to a medical patch which is configured to penetrate the medical substance to the users skin.

According to some embodiments of the present invention the computer modules include dosing module for regulating the penetration of the medical substance based on detected health condition and predefined dosing rules based on users'/patients' medical profile and/or doctor's prescription.

According to some embodiments of the present invention the smart phone sensor data comprises at least one of: GEO location, speed of movement, number of steps taken, activity level, and user profile data including Historic medical and/or genetic data.

According to some embodiments of the present invention the patch is attached to the eyeglasses frame at the distal end of the temple tip configured to be attached to skin behind the user's ear.

According to some embodiments of the present invention the patch and the reservoir are connected by a pipe using an actuator valve.

According to some embodiments of the present invention the patch is not part of the eyeglasses frame having wireless communication module, configured to be attached to different parts of the body. (and configured to submit a substance dosage calculated by the system to be suitable for the treatment of the user in real time) According to some embodiments of the present invention the audio sensor or vibration data is analyzed for testing at least one of: breath, speech properties, breath pattern volume, air flow, changes in lungs volume.

According to some embodiments of the present invention the humidity sensor data is analyzed for testing at least one of: sweating, conductivity Electrodermal activity, GSR.

According to some embodiments of the present invention wherein the monitoring application further using raw measurement data of an additional wearable device, wherein the wearable device include at least one: smart watch, in-ear device.

According to some embodiments of the present invention the system further comprising a second Replaceable elastic or semi elastic temple tip, design to be connected to the second end frame of existing eyeglasses, said temple tip including at least one optic sensor, CPU, memory, energy source and communication module.

According to some embodiments of the present invention the system further including energy management module of the two energy sources, based on usage of the sensors at each temple tip.

According to some embodiments of the present invention the monitoring module includes analysis of sweat, body temperature. GRP and heart rate for applying polygraph-based algorithm. (need to describe what exactly it does) According to some embodiments of the present invention wherein the AI learning algorithm include a deep learning algorithm configured to for detection and prediction of CVD conditions, wherein the deep learning is based on user profile inputs, user feedback received in real time which are compared with measured health data from the system sensors.

According to some embodiments of the present invention further comprising an actuator for controlling the dosing of the medical substance.

According to some embodiments of the present invention the system further comprising an elastic band connecting the two temple tips enabling to keep the temple tip close to the skin.

According to some embodiments of the method present invention further comprising the steps of: Receiving blood measured parameter from the patch, checking if blood measured parameters exceeds predefined values, sending an alert to the user.

According to some embodiments of the present invention the method further comprises applying an AI model using sensor data of blood measured parameters with at least on of BCG, PPG, ECG and PTT to identify patient cardiologic activity, cardiovascular disorder, based client profile and sensor data from the mobile application.

### BRIEF DESCRIPTION OF THE INVENTION

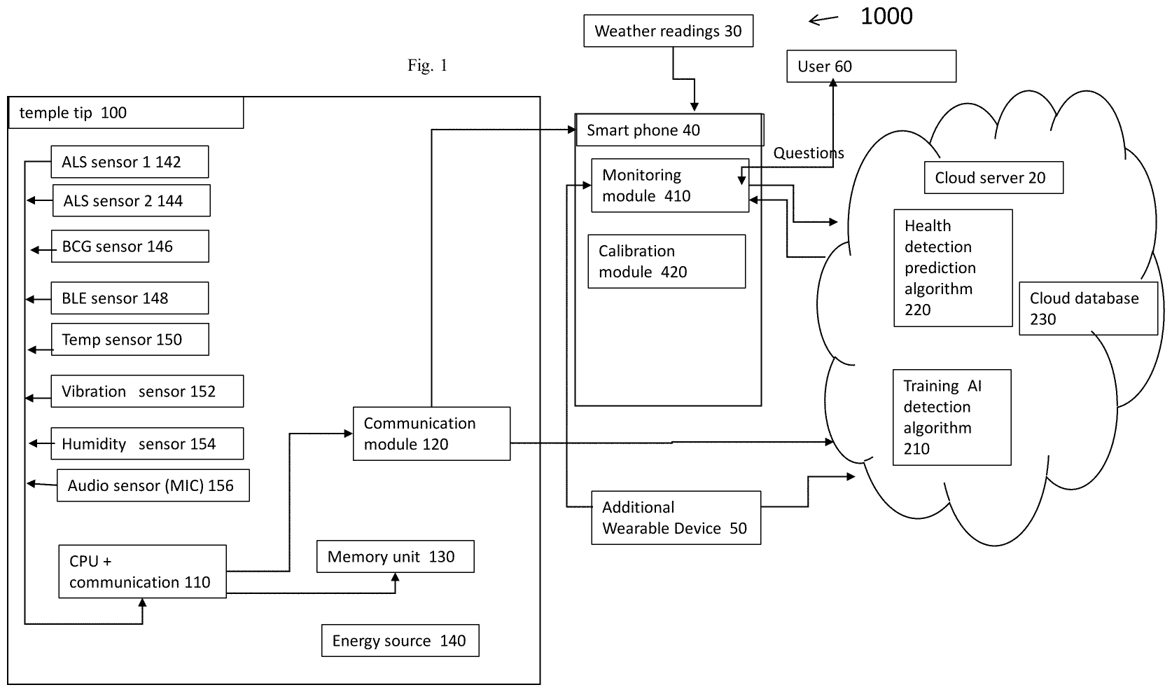

FIG. 1 illustrates a schematic view of a health monitoring system for monitoring vital signs of a user in accordance with some embodiments of the present invention.

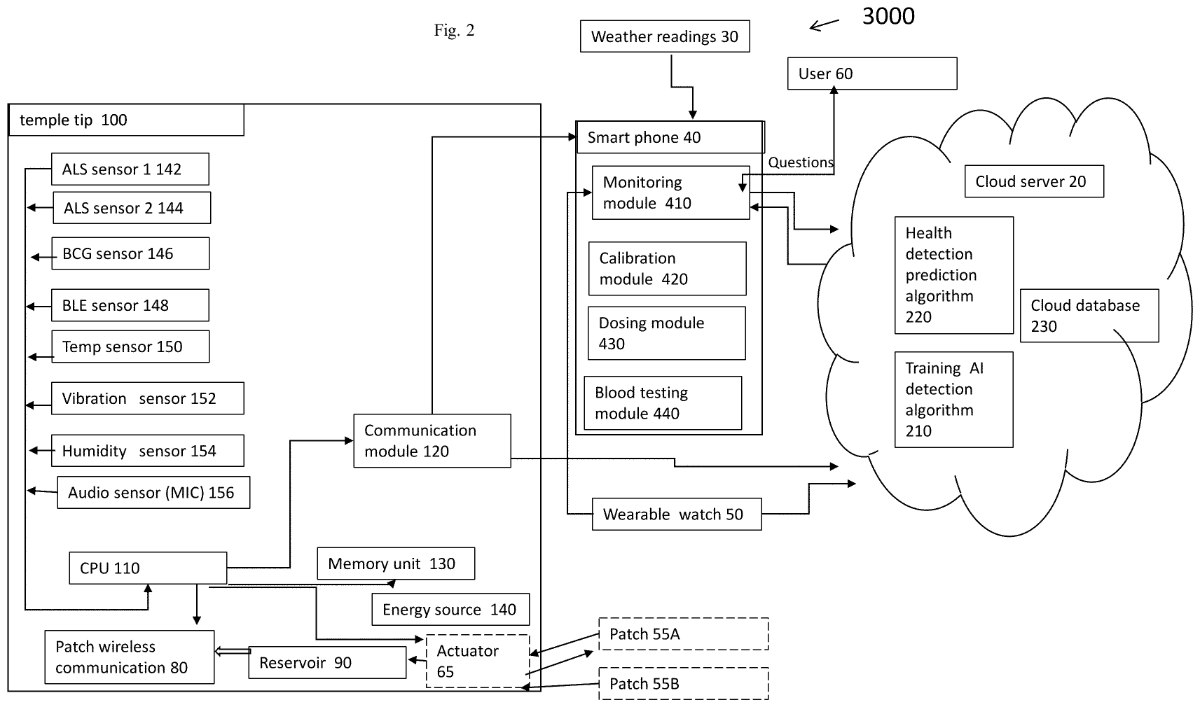

FIG. 2 illustrates a health monitoring system for monitoring vital signs and for providing controllable treatment to the user in accordance with some embodiments of the present invention.

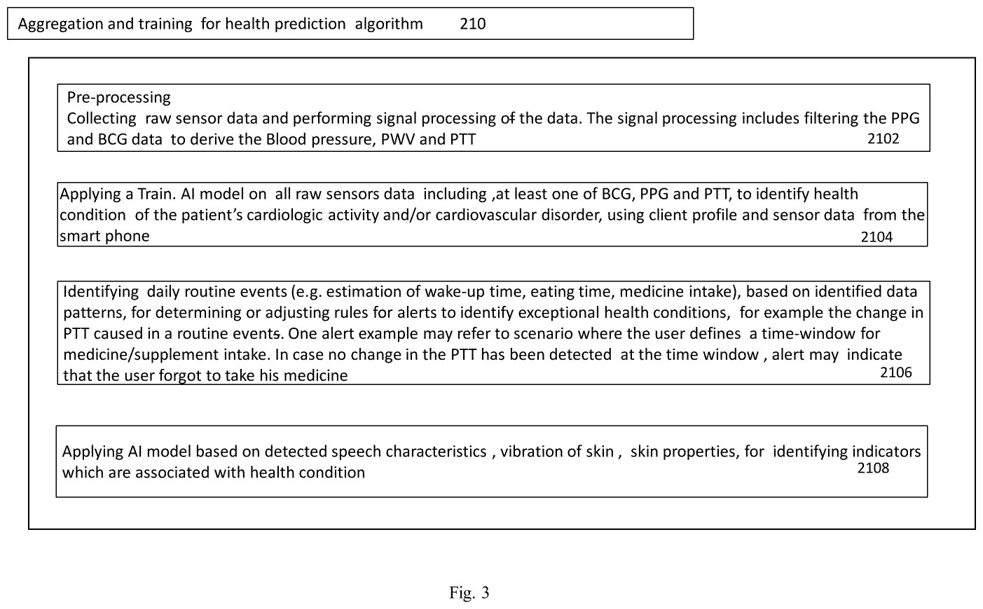

FIG. 3 illustrates a health monitoring system for alerting health condition of a user in accordance with some embodiments of the present invention

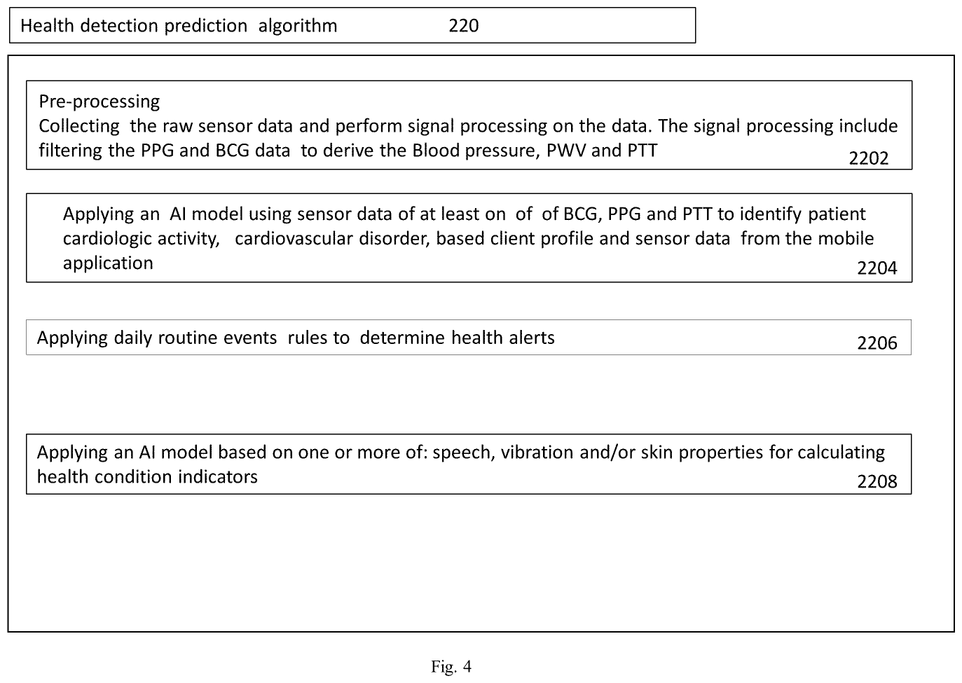

FIG. 4 illustrates health detection prediction algorithm in accordance with some embodiments of the present invention.

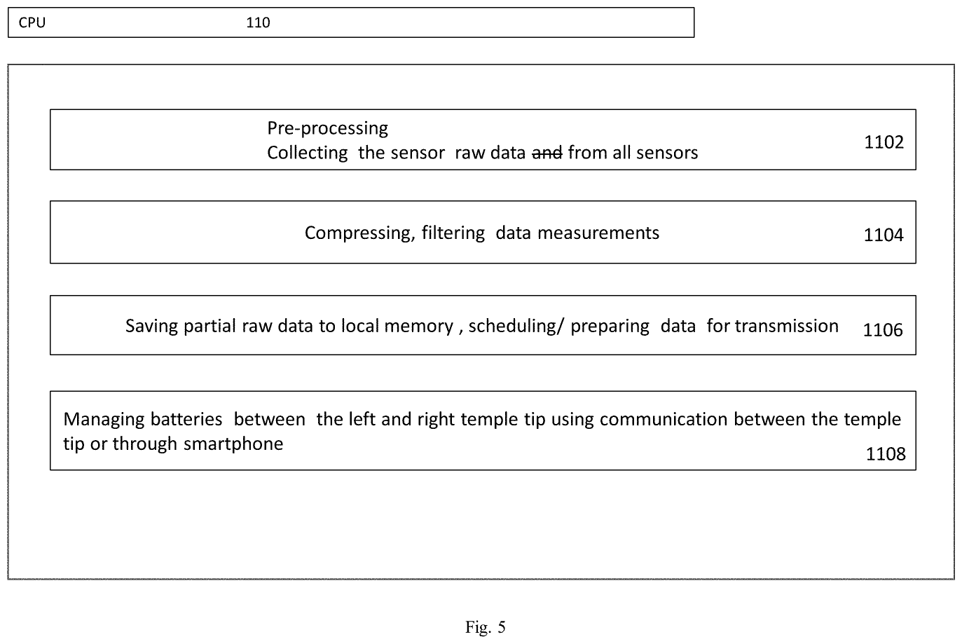

FIG. 5 illustrates the CPU activities in accordance with some embodiments of the present invention.

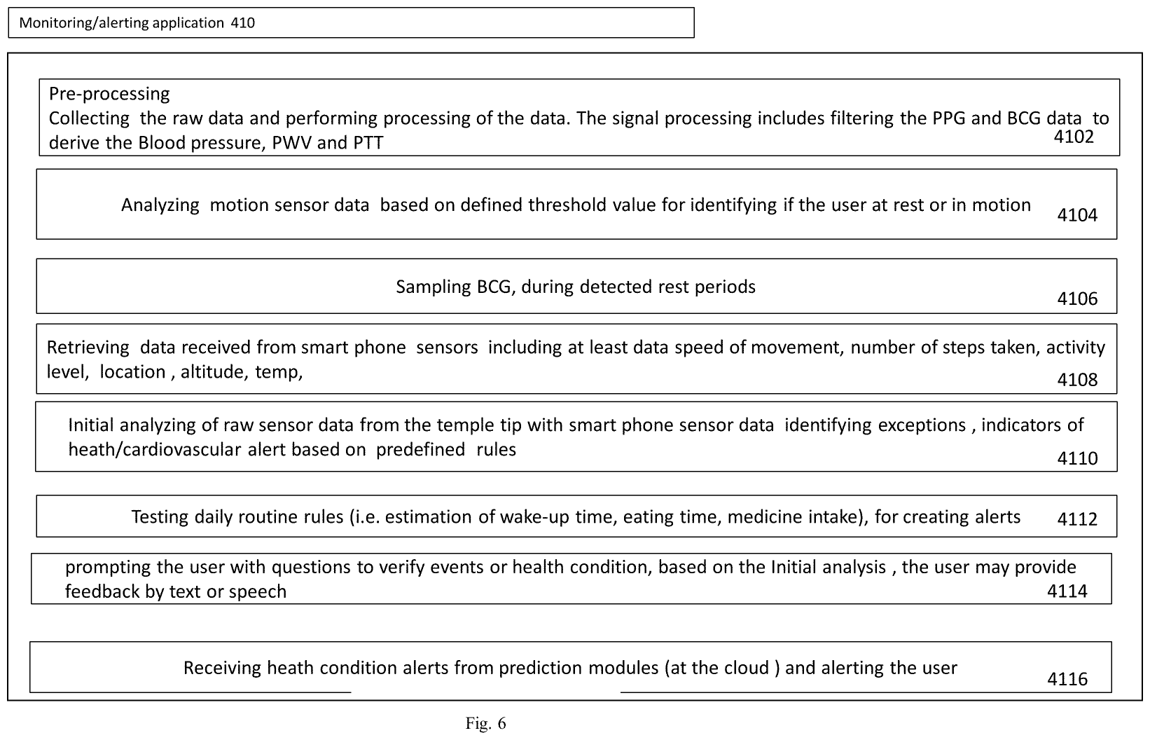

FIG. 6 illustrates the monitoring/alerting application in accordance with some embodiments of the present invention.

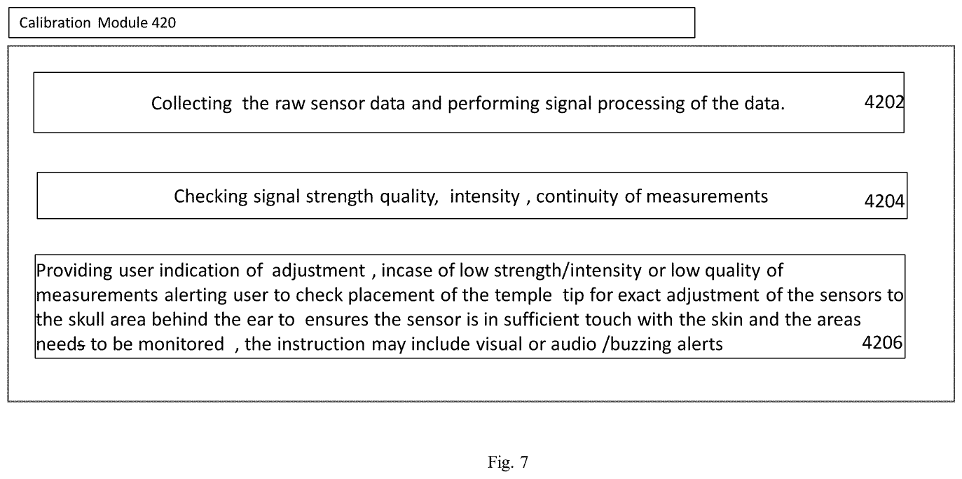

FIG. 7 illustrates the calibration module in accordance with some embodiments of the present invention.

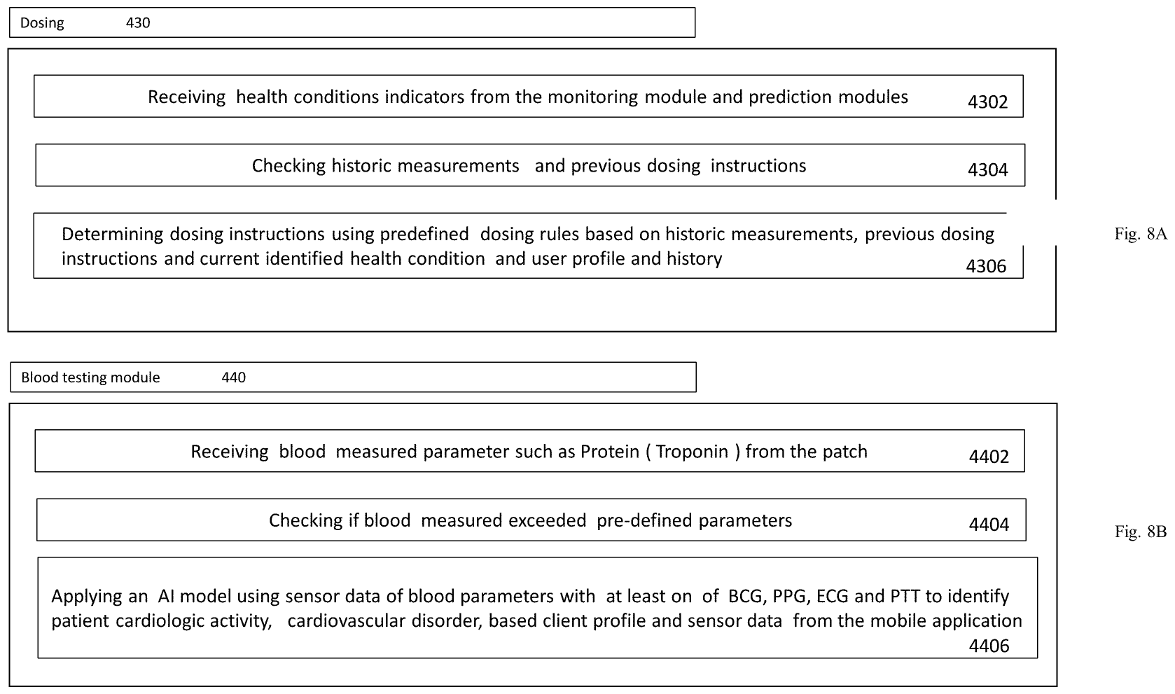

FIG. 8A illustrates the dosing module in accordance with some embodiments of the present invention.

FIG. 8B illustrates the blood testing module in accordance with some embodiments of the present invention.

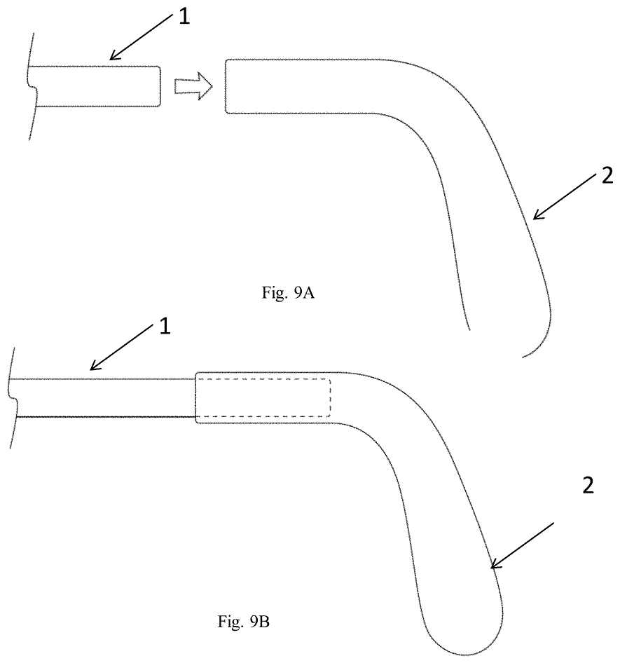

FIG. 9A illustrates an example of the temple tip in integration with glasses eyeglasses frame in accordance with some embodiments of the present invention.

FIG. 9B illustrates an example of the temple tip in integration with glasses eyeglasses frame in accordance with some embodiments of the present invention.

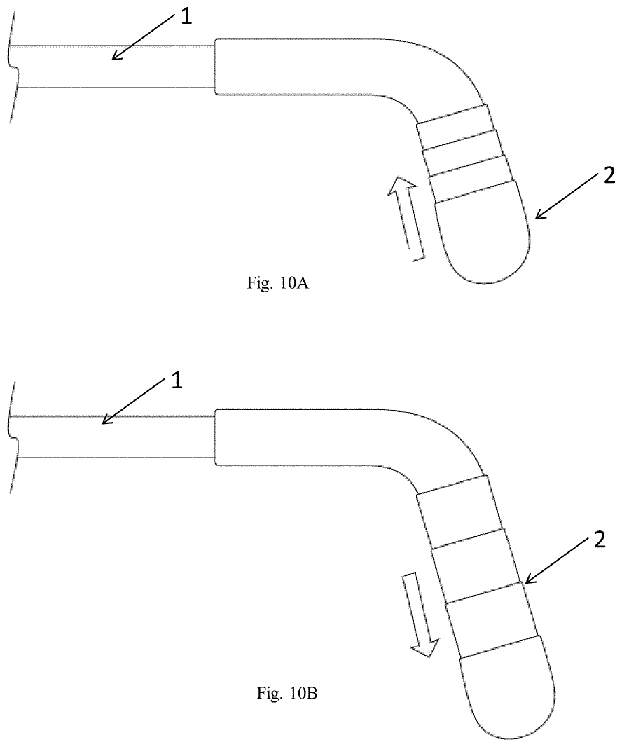

FIG. 10A illustrates an example of the temple tip in integration with glasses eye glasses frame having telescopic mechanism in accordance with some embodiments of the present invention.

FIG. 10B illustrates an example of the temple tip in integration with glasses eye glasses frame having telescopic mechanism in accordance with some embodiments of the present invention.

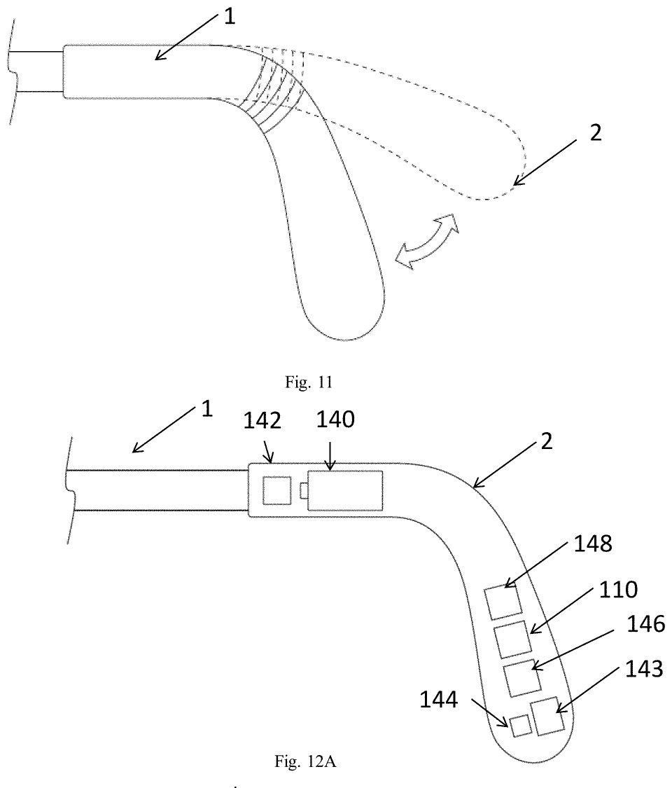

FIG. 11 illustrates an example of the temple tip in integration with glasses eyeglasses showing possible elastic movement of the temple tip in accordance with some embodiments of the present invention.

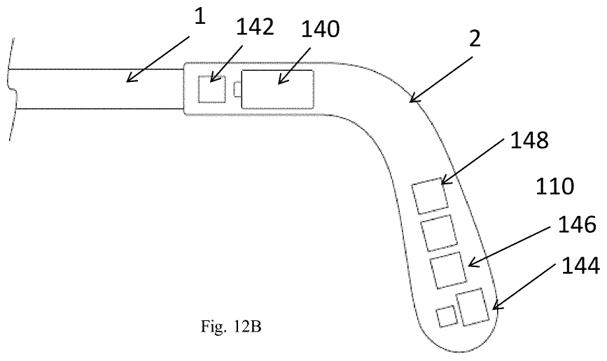

FIG. 12A illustrates an example of the temple tip in integration with eyeglasses frame with inner components of the temple tip in accordance with some embodiments of the present invention.

FIG. 12B illustrates an example of the temple tip in integration with eyeglasses frame with inner components of the temple tip in accordance with some embodiments of the present invention. ???

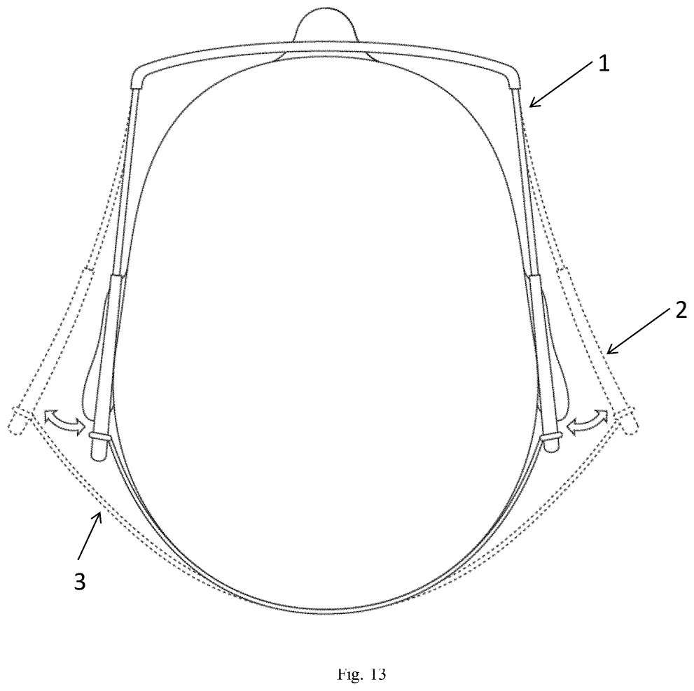

FIG. 13 illustrates an example of the temple tip in integration with eyeglasses showing possible elastic movement of the temple tip in accordance with some embodiments of the present invention.

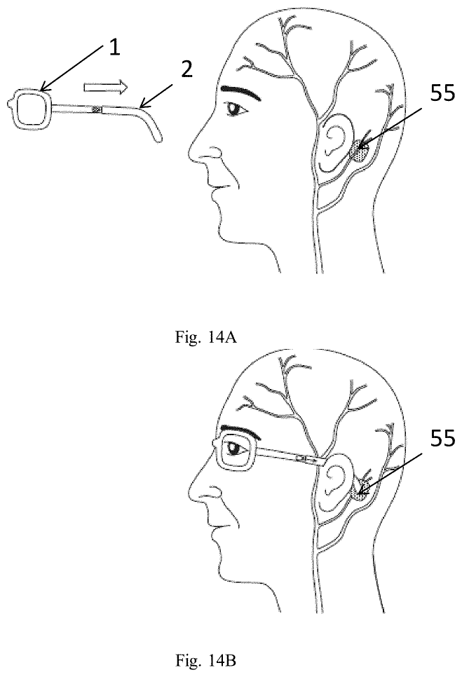

FIGS. 14A&14B illustrate an example of the temple tip in integration with eyeglasses placement on user head in accordance with some embodiments of the present invention.

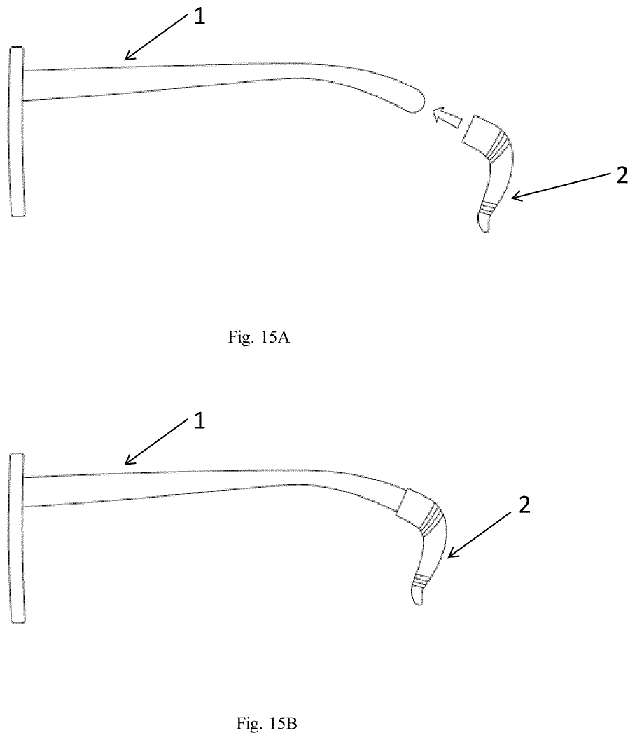

FIGS. 15A&15B illustrate an example of the temple tip in integration with eyeglasses frame, where temple tip is placed on top of original temple tip of the eyeglasses frame in accordance with some embodiments of the present invention.

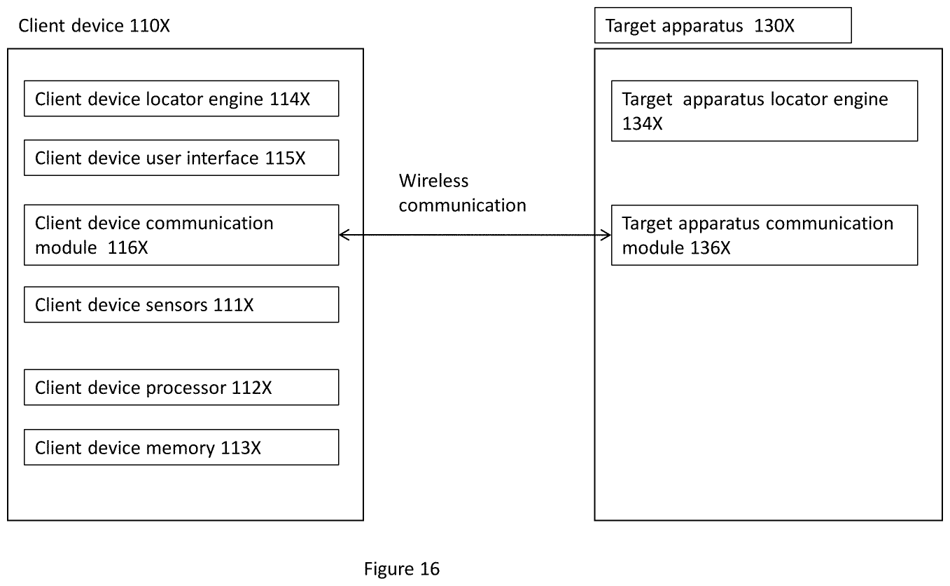

FIG. 16 presents the client device 110 and the target apparatus 130 according to one embodiment of the present invention.

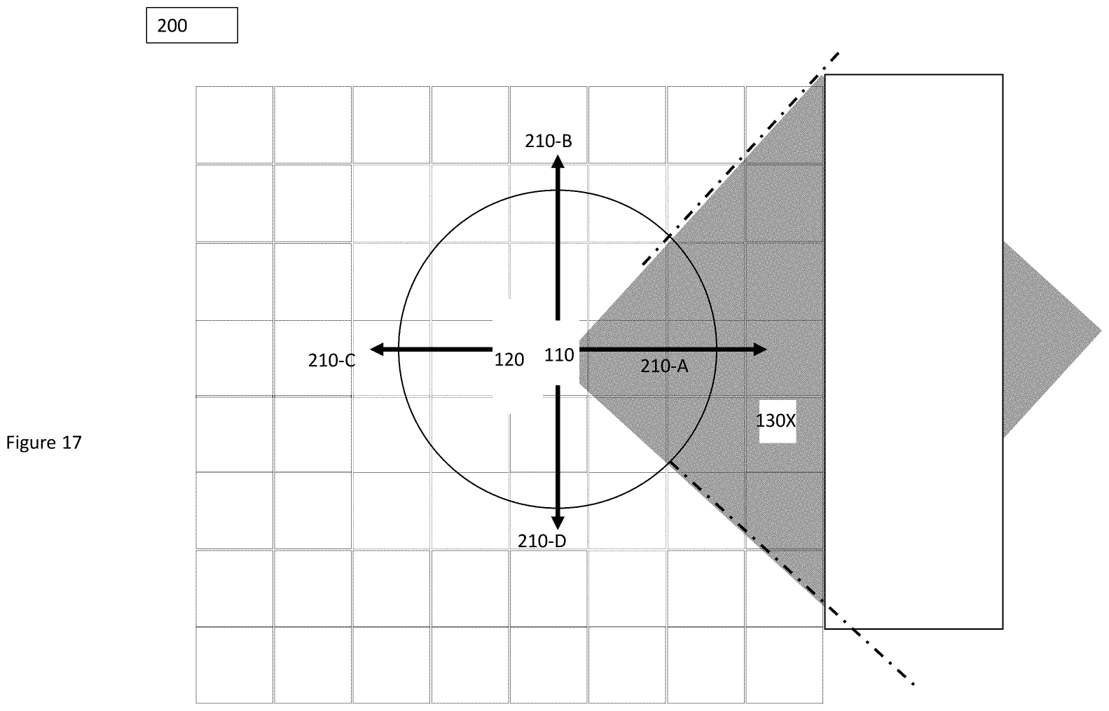

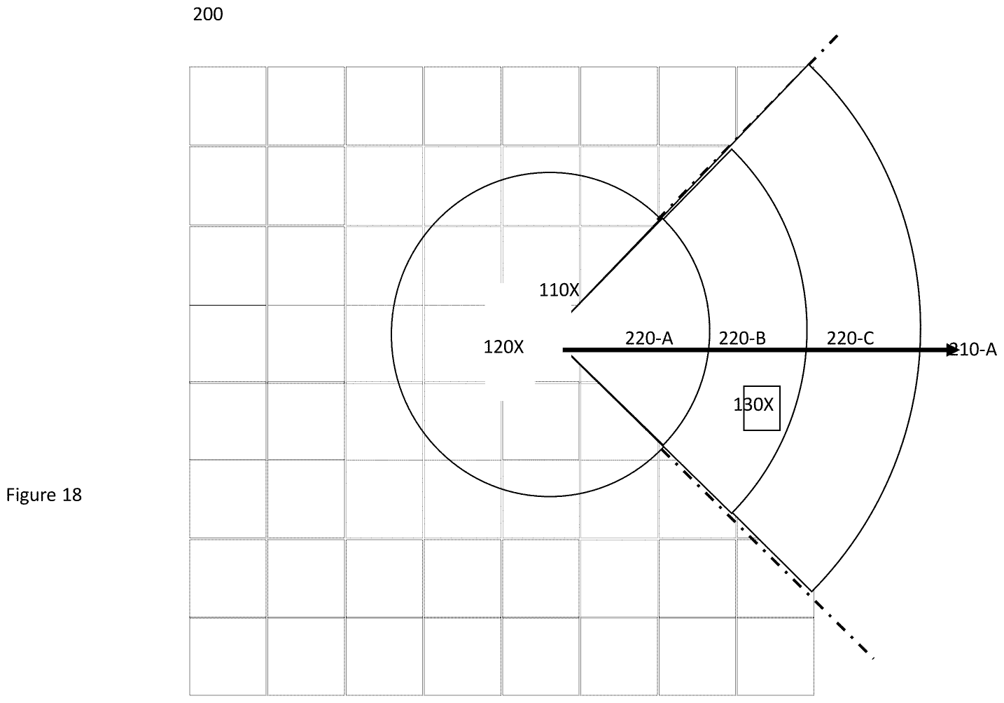

FIGS. 17 and 18 jointly present a schematic diagram of the search environment 200 in which a process of target apparatus location is carried out according to one embodiment of the present invention.

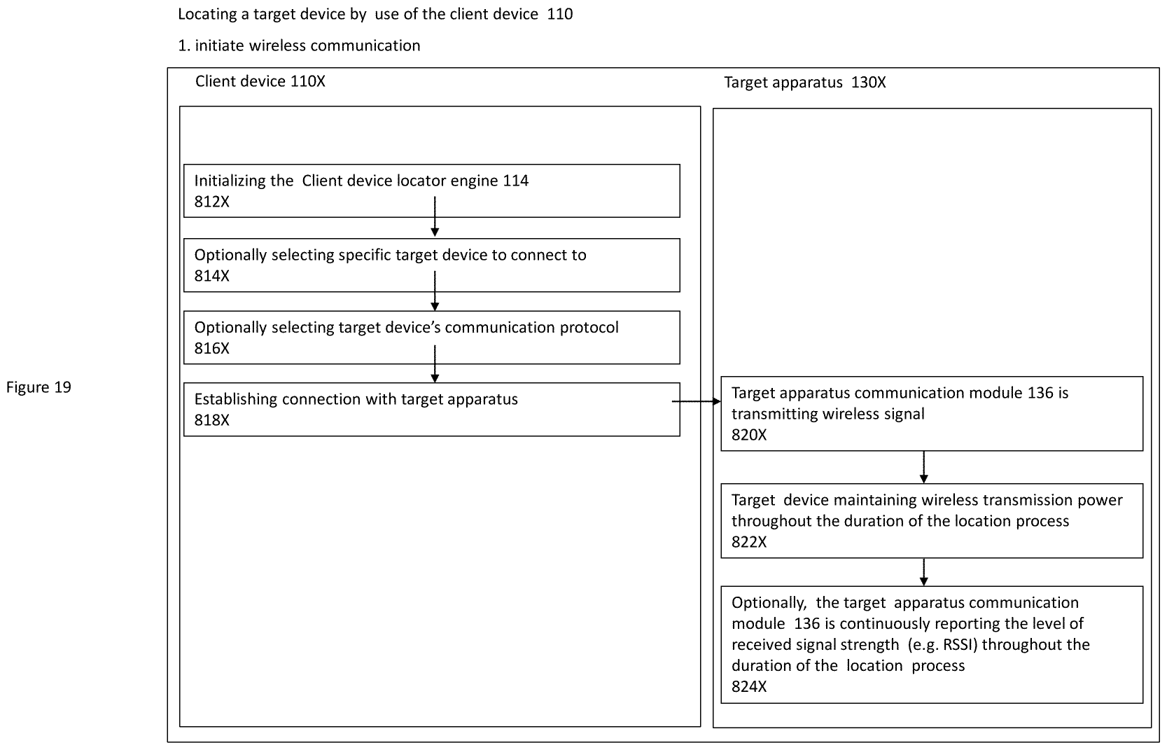

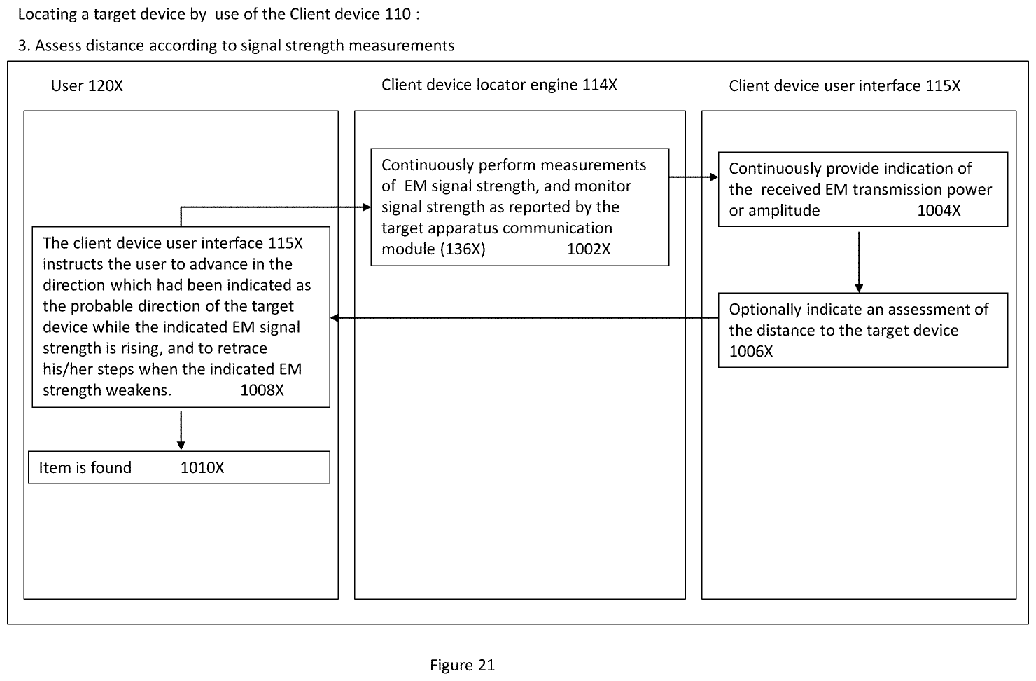

FIGS. 19, 20 and 21 jointly present a flow diagram, depicting the three stages of the process of target apparatus location in reference to a polar coordinates system, according to one embodiment of the present invention.

### DETAILED DESCRIPTION OF THE FIGURES

FIG. 1 illustrates a schematic view of a health monitoring system 1000 for monitoring vital signs of a user in accordance with some embodiments of the present invention.

In accordance with some embodiments of the present invention, the health monitoring system 1000 comprises at least one replaceable elastic/semi elastic temple tip 100 (also referred to as temple guard) designed to be connected to one end or part of a frame of existing eyeglasses and monitoring application.

In accordance with some embodiments of the present invention, the replaceable elastic temple tip is configured to be bent in towards and/or inward the designated area of measurement, also incorporating a function with which the temple tip can be adjusted in length, height and depth to provide optimal measurements as well as optimal wearing comfort of the eyewear frame taking into account optical requirements.

In accordance with some embodiments of the present invention, the replaceable elastic temple tip is configured to move backward or forward, enabling the user to adjust the temple tip position to achieve optimal measurement results (must be mentioned that the App will notify the user/patient if the temple tip needs to be adjusted).

In accordance with some embodiments of the present invention, the temple tip 100 comprises multiple components including sensors such as at least two optical sensor 142 (such ALS sensor (ambient light sensor) sensor 144, ballistocardiograph (BCG) sensors (such as accelerometer sensor 146, BLE sensor 148, a temperature sensor 150, a vibration sensor 152, a humidity sensor 154, and an audio sensor 156.

The temple tip further comprises a CPU with communication capabilities 110, a communication module 120, memory unit 130, and an energy source 140.

The health monitoring system 1000 further comprises a monitoring application implemented on at least one computerized monitoring module 410 for aggregating raw measurement data, analyzing raw data using health-oriented AI model for prediction of health conditions and providing alerts to the user or caregiver based on said prediction.

The AI model integrates data received by the optical sensor(s) together with data received from the other sensors of the system

The AI model may be implemented by deep learning algorithm for improving detection and prediction of CVD conditions. The Deep learning algorithm may use user profile inputs (i.e., Age, smoking, weight, history of CVD in the family etc.) as well as user feedback received in real time (i.e., physical activity, food & drug intake, etc.) and compare with measured health data from the system sensors (i.e., PTT, PVW, blood saturation, QRS interval) to improve the analysis of the current user status.

In accordance with some embodiments of the present invention, a health detection prediction algorithm 220, a training Al detection algorithm 210 and a cloud database 230 are implemented on a cloud server 20.

In accordance with some embodiments of the present invention, computerized (and data aggregation engine) modules such as the monitoring module 410, calibration module 420, and dosing module 430 are implemented on a smart phone, wherein the smart phone modules provide sensor data of the smart phone and user profile, enabling integrated analysis of temple tip sensor data and smart phone sensor data and profile data.

In accordance with some embodiments of the present invention, the smartphone is used as a vice-versa data communication platform which displays personalized data to the user, displays personalized questions to the user (which are a result of data aggregation done on the cloud server).

In accordance with the present invention, the smartphone usages are: transfer data from the device to the cloud platform to communicate with the patient (through the dedicated Smart phone app), collect the location of the patient, show relevant data to the patient (after analysis on the cloud), platform to communicate with a caregiver in case of emergency (video or phone call), use the smart phone earpiece to recording the speech of the patient on different daily situations and detect any breath shortness, use the smart phone camera to measure blood pressure on the finger (in special cases)—use the camera to take a picture (there are modules to detect stroke by the face)—, track the steps made by the patient (sport activity and steps counter as indicator of the patients activity level).

In accordance with some embodiments of the present invention, the smartphone further comprising calibration module for checking signal quality parameters for alerting user of adjusting position of the temple tip for improving signal quality. In accordance with some embodiments of the present invention, the smartphone further includes a dosing module for regulating the penetration of the medical substance based on detected health condition and predefined dosing rules based on users'/patients' medical profile and aggregated data from the cloud server.

In accordance with some embodiments of the present invention, the smart phone sensor data comprises at least one of: GEO location, speed of movement, number of steps taken, activity level, and user profile data including Historic medical and/or GEO Location data.

In accordance with some embodiments of the present invention, the audio sensor or vibration data is analyzed for testing at least one of: breath frequency, breath speech properties, breath pattern, breath volume, breath frequency, air flow, changes in lungs volume

In accordance with some embodiments of the present invention, the humidity sensor data is analyzed for testing at least one of: sweating, conductivity Electrodermal activity, GSR. (there should be mentioning of polygraph capabilities as a result of these measurements)

In accordance with some embodiments of the present invention the health monitoring system comprises Polygraph algorithm-based measurements of sweat, temperature and heart rate or GSR (Galvanic skin response) data.

The polygraph algorithm sets a threshold value for skin conductivity, heart rate and blood pressure/blood flow pattern and according to these thresholds the system is able to detect if the person says the truth. The threshold setting can be defined in comparison to the values when the person is resting and not under test.

In accordance with some embodiments of the present invention, the monitoring application further gathers data of an additional wearable device (such as a smart watch), using sensor data form different part of body to improve the analysis.

In accordance with some embodiments of the present invention, the health monitoring system further comprising a second replaceable elastic or semi elastic temple tip designed to be connected to the second end frame of existing eye glasses, said temple tip including one optic sensor, acceleration sensor, CPU, memory, energy source and communication module.

In accordance with some embodiments of the present invention, the health monitoring system further includes an energy management module of the two energy sources, based on usage of the sensors at each temple tip.

In accordance with some embodiments of the present invention the health monitoring system further includes an actuator 65 for controlling the dosing of the medical substance. The actuator is controlled by the CPU 110.

One of the main roles of the accelerometer sensor is to measure the BCG (Ballistocardiography). The BCG measurements will be translated to an ECG signal. The BCG signal (after processing to ECG signal) can detect abnormal activities (i.e. abnormal heart rhythm) such as Atrial fibrillation (AF), furthermore, the BCG measurements together with other measurements provided by sensors of the Temple tip may create more accurate measurements by comparing of multiple measurements by the system with each other, for example comparing of exceptional body temperature with exceptional sweat measurement and/or with exceptional PWV(pulse wave velocity).

According to some embodiments the system can use separate sensors in the two separate temples of the Eyeglasses:

Optionally the second temple tip battery and sensors may serve as backup for the first, such as, in case, the first side's battery is about to deplete a signal can be sent to the second side in order to overtake the measurements until the battery is charged by the user.

According to another option second temple tip battery and sensors may serve for measurement verification in case of exceptional measurement beyond predefined threshold is detected by the first temple tip sensors, the measurement can be verified by a separate and independent measurement of the sensors in the second temple for result verification purposes.

According to some embodiments of the present invention, the users body temperature is used as an additional parameter to detect CVD and will improve the analysis of the other sensors measurements. Body temperature may be compared to the current outside temperature which is provided by the users Smartphone. The comparison of both temperatures will act as an additional way to verify reasons for changes in body temperature. In another embodiment the system measuring skin conductivity may be compare this measurement with the outside humidity in the user's location to provide a more exact analysis of the user's skin conductivity.

The smartphone application enable to transfer data from the temple tip device to the cloud, collect the location of the patient and show relevant analyzed data to the patient.

Optionally the smartphone application may include one or more of the following features:
enable the user to communicate with the caregiver in the case of an emergency (video or phone call),
use the smart phone earpiece to recording the speech of the patient on different daily situations and detect any breath shortness,
use the smart phone camera to measure blood pressure on the finger (in special cases),
use the camera to take a picture using algorithm to detect stroke by analyzing face image
track the steps made by the patient to identify general and sport activity.

FIG. 2 illustrates a health monitoring system 3000 for alerting health condition of a user in accordance with some embodiments of the present invention.

As seen in the figure, the Replaceable elastic temple tip further includes a reservoir which maintains medical substance, connected to a metical patch which is configured to penetrate the medical substance under the user skin.

The patch is attached to the eyeglasses frame at the distal end of the temple tip configured to be attached to skin behind the user's ear.

In accordance with some embodiments of the present invention, the patch 55 is not part of the eyeglasses frame having wireless communication module, configured to be attached to different parts of the body.

In accordance with some embodiments of the present invention, the patch 55 includes a blood measurements unit for measuring Troponin levels in the blood, using blood measuring techniques such as are used in glucose measuring sensors In accordance with some embodiments the smart phone further including dosing module 430 for controlling the activation of the patch for doing medication.

In accordance with some embodiments the smart phone (40) further includes a blood testing module 430 for analyzing blood measurements for identification of medical conditions of the patient

FIG. 3 illustrates aggregation and training for health prediction algorithm 210 in accordance with some embodiments of the present invention.

In accordance with the present invention, in a Pre-processing stage, the raw data is collected and signal processing is performed on the data. The signal processing includes filtering the PPG and BCG data to derive the blood pressure, PWV and PTT (2102).

In a training stage, training AI model on all sensor data including at least of BCG, PPG and PTT to identify health condition of patient cardiologic activity, cardiovascular disorder using client profile and sensor data from the smart phone (2104).

In accordance with some embodiments of the present invention, daily routine events (i.e., estimation of wake-up time, eating time, medicine intake) data pattern are identified.

Based on the identified data pattern, rules/alerts to identify exceptional health condition are determined. For instance, the change in PTT caused by routine events. One alert example, in case the PTT value has not changed (no events occurred) in the time window set by the customer for medicine intake (2106).

Further in the training stage, training AI model is based on detected speech characteristics, vibration of skin, skin properties, for identifying indicators which are associated with health condition (2108).

FIG. 4 illustrates health detection prediction algorithm 220 in accordance with some embodiments of the present invention.

In accordance with some embodiments of the present invention, in a pre-processing stage raw data is collected and signal processing is performed on the data. The signal processing includes filtering the PPG and BCG data to derive the blood pressure, PWV and PTT (2202).

In the next stage, an Al model is applied on data received from BCG, PPG, and PTT sensors. Based on the sensory data as well as client data received via the mobile application, patient cardiologic activity and cardiovascular disorder are identified (2204).

Daily routine events rules are applied daily to determine health alerts (2206). Al speech/vibration/skin properties model is applied for analyzing speech, vibration skin properties and for calculating indicators for health condition (2208).

FIG. 5 illustrates the CPU 110 activities in accordance with some embodiments of the present invention.

As seen in the figure, at a pre-processing stage, the CPU collects raw measurement data from all sensors (1102).

The CPU compresses the data (1104) and optionally filters the data.

The CPU saves partial raw data to local memory and schedules/prepares data for transmission (1106).

The CPU manages batteries between the left and right temple tip using communication between the temple tip or through a smartphone (1108).

FIG. 6 illustrates the monitoring/alerting application 410 in accordance with some embodiments of the present invention.

In a pre-processing stage, raw data is collected and signal processing is performed on the data. The signal processing includes filtering the PPG and BCG data to derive the blood pressure, PWV and PTT (4102).

Data received from a motion sensor is analyzed based on a predefined threshold value for identifying whether the user is at rest or in motion (4104).

During detected rest periods, BCG is sampled (4106).

Data received from smart phone sensors including at least speed of movement, number of steps taken, activity level, location, altitude, temp is retrieved (4108). An initial analysis of sensor raw data from the temple tip as received via a smart phone for identifying exceptions, indicators of heath/cardiovascular alert based on predefined rules (4110).

Daily routine rules are tested, i.e., estimation of wake-up time, food intake time, medicine intake in order to create relevant alerts (4112).

Based on Initial analysis prompting the user with questions to verify events or health condition, the user may provide feedback by text or speech. The user answers can be used as input to the training module (4114);

Health condition alerts are received from prediction modules (at the cloud) for alerting the user (4116).

FIG. 7 illustrates the calibration module 420 in accordance with some embodiments of the present invention.

In accordance with some embodiments of the present invention, raw data is collected and signal processing is performed on the data (4202).

Properties such as signal strength quality, intensity, continuity of measurements are checked (4204).

User indication of adjustment is provided so that in case of a low strength/intensity or low quality of measurements the user is alerted to check the placement of the temple tip for adjustment the sensors to the skull area behind the ear to ensure the sensor is in sufficient touch or distance to or from the skin and the areas needed to be monitored. Instructions may be provided via visual or audio/buzzing alerts (4206).

FIG. 8A illustrates the dosing module 430 in accordance with some embodiments of the present invention.

Health condition indicators are received from the monitoring module 410 and the health detection prediction algorithm 220 (4302).

Checking historic measurements and previous dosing instructions (4304).

Dosing instructions are determined using predefined dosing rules based on current identified health condition, historic measurements and previous dosing instructions and user profile and history (4306).

FIG. 8B illustrates the blood testing module 440 in accordance with some embodiments of the present invention.

Receiving blood measurement parameter such as Protein (Troponin) from the patch (4402).

Checking if blood measurement exceeds pre-defined parameters, sending alert to the user (4404).

Applying an AI model using sensor data of blood parameters with at least one of BCG, PPG, ECG and PTT to identify patient cardiologic activity, cardiovascular disorder, based client profile and sensor data from the mobile application (4306).

FIG. 9A illustrates an example of the temple tip in integration with eyeglasses frame in accordance with some embodiments of the present invention.

FIG. 9B illustrates an example of the temple tip in integration with eyeglasses frame in accordance with some embodiments of the present invention.

FIG. 10A illustrates an example of the temple tip in integration with eyeglasses frame having telescopic mechanism in accordance with some embodiments of the present invention.

FIG. 10B illustrates an example of the temple tip in integration with glasses eye glasses frame having telescopic mechanism in accordance with some embodiments of the present invention.

FIG. 11 illustrates an example of the temple tip in integration with eyeglasses showing possible elastic movement of the temple tip in accordance with some embodiments of the present invention.

FIG. 12A illustrates an example of the temple tip in integration with glasses eyeglasses frame with inner components of the temple tip in accordance with some embodiments of the present invention.

FIG. 12B illustrates an example of the temple tip in integration with glasses eye glasses frame with inner components of the temple tip in accordance with some embodiments of the present invention.

FIG. 13 illustrates an example of the temple tip in integration with eyeglasses showing possible elastic movement of the temple tip in accordance with some embodiments of the present invention. according to this embodiment, the temple tip can move towards or away to/from the user's skin. Optionally an elastic ribbon 3 can be stretched between the left and right temples time to maintain the temple tip sensors close contact to the skin.

FIGS. 14A&14B illustrate an example of the temple tip in integration with eyeglasses placement on a user's head in accordance with some embodiments of the present invention. These figures visualize the placement of the temple tip on the user's head showing the artery location. These figures further show the patch 55 placement on the user's head.

FIGS. 15A&15B illustrates an example of the temple tip in integration with glasses eyeglasses placement accordance with some embodiments of the present invention. According to this embodiment, the temple tip 2 is places over the original temple tip of eye glasses frame 1.

FIG. 16 presents the client device 110X and the target apparatus 130X according to one embodiment of the present invention.

The client device 110X may be implemented on any suitable device (e.g. a Smartphone or a tablet PC), fully or partially.

The client device locator engine 114X utilizes the resources of the client device 110X (e.g. client device processor 112X and client device memory 113X) to implement the process of target apparatus 130X location, as required and configured by the user. The client device locator engine 114X may be implemented as any combination of hardware and software, and is not restricted to any specific architecture or platform.

According to some embodiments of the present invention, the client device locator engine 114X is implemented as a mobile application, providing a user interface 115X for system configuration and target apparatus 130X location indications.

According to some embodiments of the present invention, the client device locator engine 114X utilizes additional sensors 111X embedded within the client device 110X platform, such as inertial sensors, to enhance the presented target apparatus location during the location process.

The client device communication module 116 implements any type of wireless protocol (e.g., Wi-Fi, Zigbee, Bluetooth), to connect to the appropriate target apparatus 130X communication module 136X during the process of target apparatus location.

The target apparatus locator engine 134X manages the operation of the target apparatus' communication module 136X (e.g. protocol type, activation of wireless transmission and transmitted signal strength).

According to one embodiment of the present invention, the target apparatus locator engine 134X controls the communication module 136X in accordance with instructions it receives from the client device locator engine 114X.

FIGS. 17 and 18 jointly present a schematic diagram of the search environment 200X in which a process of target apparatus location is carried out according to one embodiment of the present invention. In this embodiment, the process of target apparatus 130X finding is performed (but not restricted to) in reference to a polar coordinate system (PCS). The client device 110X is initially located at the pole of the said PCS, and the location of the target apparatus 130X is measured and reported in reference to the client device's (110X) position.

The location process is performed in three stages:
The 1^{st} stage is a preliminary stage of initializing the client device locator engine 114X and the target apparatus locator engine 134X, and is discussed below.
The 2^{nd} stage is depicted in FIG. 17. In this stage, the client device 110X divides the area surrounding the client device 110X to several search sectors. The said search sectors may form the shape of a pie slice (e.g. in outdoor environments), or a polygon (e.g. when performing the search within closed quarters).
The client device 110X performs signal strength measurements (210-A, 210-B, 210-C, 210-D) per each of the search sectors. By this method, the client device 110 obtains the probable direction to the target apparatus 130X (e.g. direction of 210-A). This stage is further elaborated below.
The 3^{rd} stage is depicted in FIG. 18. In this stage, the target apparatus 130X is traced by advancing the client device 110 along the said probable direction (e.g. 210-A), through areas pertaining to different levels of received signal strength (220-A, 220-B, 220-C), until the target apparatus is found. This stage is further elaborated below.

FIGS. 19, 20 and 21 jointly present a flow diagram, depicting the three stages of the said process of target apparatus location in reference to a polar coordinates system, according to one embodiment of the present invention.

FIG. 19 shows the initialization of the client device locator engine 114 and the target apparatus locator engine 134.
The client device locator engine 114X is activated, and the client device user interface 115X is made available for a user to operate the system (step 812X).
According to one embodiment, the client device 110X enables the user to select a specific target apparatus 130X to connect to from a given list of target apparatuses 130X (step 814X).
According to one embodiment, the client device 110X enables the user to select a specific wireless communication protocol (e.g., RFID, Wi-Fi, Bluetooth) to connect to the said target apparatus 130 (step 816).
The client device 110X connects with the said target apparatus (step 818X).
According to one embodiment, the target apparatus communication module 136X transmits a wireless signal constantly or periodically, maintaining the wireless EM transmission power throughout the duration of the location process (steps 820X, 822X).
According to one embodiment, the target apparatus communication module 136 continuously reports the level of its received signal strength (e.g. RSSI) throughout the duration of the location process. This value serves as an indicator to the vicinity of the target apparatus 130 to the client device 110 (step 824X).

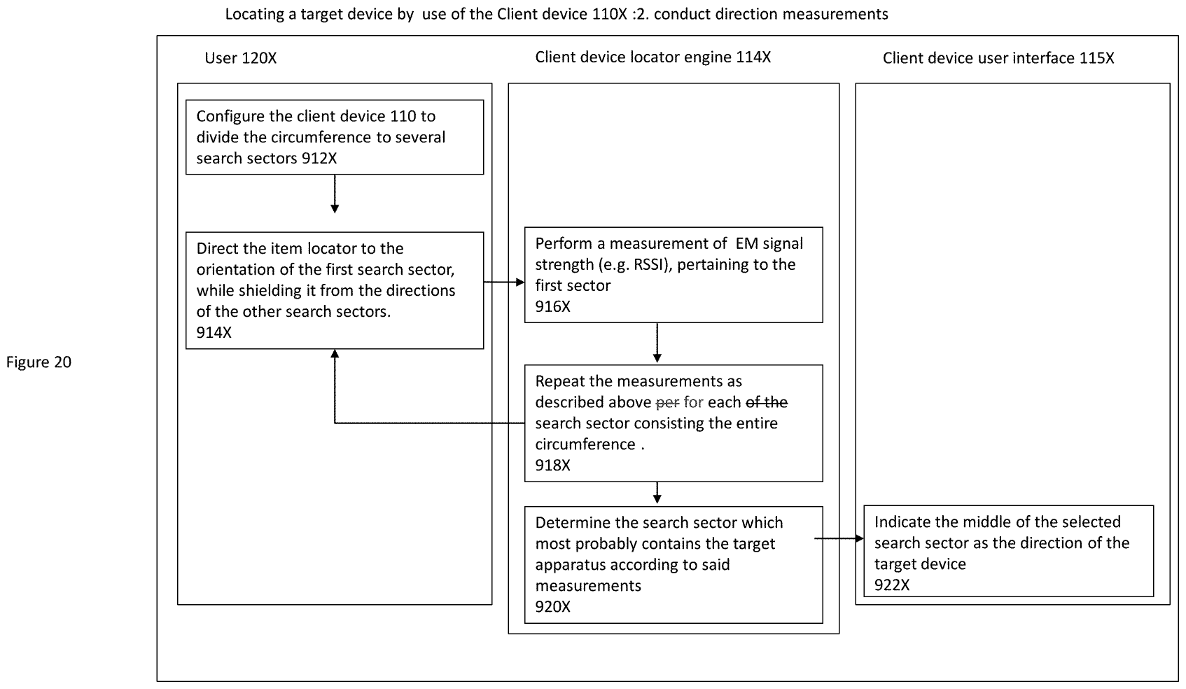

FIG. 20 presents the 2^{nd} stage of the target apparatus location process in reference to a polar coordinates' system, according to one embodiment of the present invention. This process is also graphically depicted in FIG. 17 for additional clarification.
The client device is configured to divide the area surrounding the client device 110X to several search sectors (step 912X).
The client device 110X instructs the user 120X to physically hold the client device 110 in the orientation of the first search sector, while shielding it from the directions of the other search sectors, in the following manner (step 914):
   The client device 110X is exposed in the front direction, to have a clear line of sight to the extent of the search sector 210 where the search for the target apparatus is performed.
   The client device 110X is held in close vicinity to the user's own body 120X and/or any other object (e.g. a frying pan). This diminishes the effect of multipath reception and/or refracted waves of the target apparatus transmission, and/or the effect of reception side lobes from the target apparatus 130X wireless transmission.
The client device locator engine 114X performs a measurement of EM signal strength (e.g. RSSI), pertaining to the first search sector 210X (step 916X)
These measurements are repeated as described above per each of the search sectors, constituting a complete wireless EM signal strength measurement of the client device's entire circumference (step 918X).
The client device locator engine 114X determines the search sector which contains the target apparatus according to said EM signal strength measurements (step 920X). The correct search sector may be determined according to various criteria, such as:
   The search sector bearing maximal received signal strength by the client device locator engine 114X
   The search sector opposite the one bearing minimal received signal strength by the client device locator engine 114X
   The search sector bearing maximal received signal strength by the target apparatus communication module 136X
   The search sector opposite the one bearing minimal received signal strength by the target apparatus communication module 136X
   Any combination of the above.
The client device user interface 115 indicates the selected search sector as the direction of the target device, e.g. 210-A (step 922X).

FIG. 20 presents the 3^{rd} stage of the target apparatus location process in reference to a polar coordinates' system, according to one embodiment of the present invention. This process is also graphically depicted in FIG. 18 for additional clarification.
The client device locator engine 114X continuously (step 1002X):
   Performs measurements of the EM signal strength (transmitted by the target apparatus communication module 136X), and
   Optionally monitors the signal strength reported by the target apparatus (transmitted by the client device communication module 116X).
The client device user interface 115X continuously provides indication of the received EM signal strength (step 1004X) and optionally indicates an assessment of the distance to the target device (step 1006X).
The client device user interface 115X instructs the user 120X to advance in the direction which had been indicated as the probable direction 210-A of the target device 130X, while the indicated EM signal strength is rising, and instructs the user 120X to retrace his/her steps when the indicated EM strength weakens (step 1008X).
This process continues until the target apparatus 130X is found (step 1010X).

In the above description, an embodiment is an example or implementation of the invention. The various appearances of “one embodiment”, “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Furthermore, it is to be understood that the invention can be carried out or practiced in various ways and that the invention can be implemented in embodiments other than the ones outlined in the description above.

The invention is not limited to those diagrams or to the corresponding descriptions. For example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

The system of the present invention may include, according to certain embodiments of the invention, machine readable memory containing or otherwise storing a program of instructions which, when executed by the machine, implements some or all of the apparatus, methods, features and functionalities of the invention shown and described herein. Alternatively or in addition, the apparatus of the present invention may include, according to certain embodiments of the invention, a program as above which may be written in any conventional programming language, and optionally a machine for executing the program such as but not limited to a general purpose computer which may optionally be configured or activated in accordance with the teachings of the present invention. Any of the teachings incorporated herein may wherever suitable operate on signals representative of physical objects or substances.

Unless specifically stated otherwise, as apparent from the following discussions, it is appreciated that throughout the specification discussions, utilizing terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining” or the like, refer to the action and/or processes of a computer or computing system, or processor or similar electronic computing device, that manipulate and/or transform data represented as physical, such as electronic, quantities within the computing system's registers and/or memories, into other data similarly represented as physical quantities within the computing system's memories, registers or other such information storage, transmission or display devices. The term “computer” should be broadly construed to cover any kind of electronic device with data processing capabilities, including, by way of non-limiting example, personal computers, servers, computing system, communication devices, processors (e.g. digital signal processor (DSP), microcontrollers, field programmable gate array (FPGA), application specific integrated circuit (ASIC), etc.) and other electronic computing devices.

The present invention may be described, merely for clarity, in terms of terminology specific to particular programming languages, operating systems, browsers, system versions, individual products, and the like. It will be appreciated that this terminology is intended to convey general principles of operation clearly and briefly, by way of example, and is not intended to limit the scope of the invention to any particular programming language, operating system, browser, system version, or individual product.

It is appreciated that software components of the present invention including programs and data may, if desired, be implemented in ROM (read only memory) form including CD-ROMs, EPROMs and EEPROMs, or may be stored in any other suitable typically non-transitory computer-readable medium such as but not limited to disks of various kinds, cards of various kinds and RAMs. Components described herein as software may, alternatively, be implemented wholly or partly in hardware, if desired, using conventional techniques. Conversely, components described herein as hardware may, alternatively, be implemented wholly or partly in software, if desired, using conventional techniques.

Included in the scope of the present invention, inter alia, are electromagnetic signals carrying computer-readable instructions for performing any or all of the steps of any of the methods shown and described herein, in any suitable order; machine-readable instructions for performing any or all of the steps of any of the methods shown and described herein, in any suitable order; program storage devices readable by machine, tangibly embodying a program of instructions executable by the machine to perform any or all of the steps of any of the methods shown and described herein, in any suitable order; a computer program product comprising a computer useable medium having computer readable program code, such as executable code, having embodied therein, and/or including computer readable program code for performing, any or all of the steps of any of the methods shown and described herein, in any suitable order; any technical effects brought about by any or all of the steps of any of the methods shown and described herein, when performed in any suitable order; any suitable apparatus or device or combination of such, programmed to perform, alone or in combination, any or all of the steps of any of the methods shown and described herein, in any suitable order; electronic devices each including a processor and a cooperating input device and/or output device and operative to perform in software any steps shown and described herein; information storage devices or physical records, such as disks or hard drives, causing a computer or other device to be configured so as to carry out any or all of the steps of any of the methods shown and described herein, in any suitable order; a program pre-stored e.g. in memory or on an information network such as the Internet, before or after being downloaded, which embodies any or all of the steps of any of the methods shown and described herein, in any suitable order, and the method of uploading or downloading such, and a system including server/s and/or client/s for using such; and hardware which performs any or all of the steps of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any step described herein may be computer-implemented. The invention shown and described herein may include (a) using a computerized method to identify a solution to any of the problems or for any of the objectives described herein, the solution optionally include at least one of a decision, an action, a product, a service or any other information described herein that impacts, in a positive manner, a problem or objectives described herein; and (b) outputting the solution.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Features of the present invention which are described in the context of separate embodiments may also be provided in combination in a single embodiment.

For example, a system embodiment is intended to include a corresponding process embodiment. Also, each system embodiment is intended to include a server-centered “view” or client centered “view”, or “view” from any other node of the system, of the entire functionality of the system, computer-readable medium, apparatus, including only those functionalities performed at that server or client or node.

1. A health monitoring system for alerting health condition of a user, said system comprised of:
At least on Replaceable elastic or semi elastic temple tip, design to be connected to a one end or part of a frame of existing eye glasses, said temple tip including at least one optic sensor, acceleration sensor or ballistocardiograph (BCG) sensor, CPU, memory, energy source and a communication module;
Monitoring application implemented on at least one computerized module, for aggregating raw measurement data, analyzing raw data using health oriented AI model for prediction of health conditions and providing alerts to the user and/or entity based on said prediction.

2. The health monitoring system of claim 1 wherein said optical sensor is a light reflecting sensor or a photodiode sensor or an ambient light sensor.

3. The health monitoring system of claim 1 wherein said elastic or semi elastic temple tip further comprises of at least one additional sensor from the list of: temperature sensors, humidity sensor, vibration sensor, audio sensor, wherein the AI model is training on integration of optical sensor data and the additional sensor data.

4. The health monitoring system of claim 1 wherein the monitoring module is implemented on a cloud server.

5. The health monitoring system of claim 1 wherein the monitoring module is implemented on smart phone device.

6. The health monitoring system of claim 1 wherein the computerized modules are partly implemented on cloud server and partly on the smart phone, wherein the smart phone module provides sensor data of the smart phone and user profile, enabling integrated analysis of temple tip sensor data and smart phone sensor data and profile data.

7. The health monitoring system of claim 6 further comprising calibration module for checking signal quality parameters for alerting user of adjusting position of the temple tip for improving signal quality.

8. The health monitoring system of claim 1 wherein the Replaceable elastic temple tip is configured to be bent in toward and/or inward from the designated area of measurement, enabling to adjust in length, height and depth to provide both optimal measurements and optical usability of the eyewear frame, wherein the designated area of measurement is one of:behind ear, in front of the ear or inside the ear.

9. The health monitoring system of claim 1 wherein the replaceable elastic temple tip is configured to move backward of forward, enabling the user to adjust the temple tip position to achieve optimal measurement results by notifying the user if the temple tip needs to be adjusted.

10. The health monitoring system of claim 1 wherein the Replaceable elastic temple further includes a reservoir which includes a medical substance, connected to a medical patch which is configured to penetrate the medical substance to the users skin.

11. The health monitoring system of claim 10 wherein the computer modules include dosing module for regulating the penetration of the medical substance based on detected health condition and predefined dosing rules based on users/patients medical profile and/or doctors prescription.

12. The system of claim 6 wherein the smart phone sensor data comprises at least one of: GEO location, speed of movement, number of steps taken, activity level, and user profile data including Historic medical and/or genetic data.

13. The health monitoring system of claim 1 wherein the patch is attached to the eye glasses frame at the distal end of the temple tip configured to be attached to skin behind the user's ear.

14. The health monitoring system of claim 13 wherein the patch and the reservoir are connected by a pipe using an actuator valve.

15. The health monitoring system of claim 1 wherein the patch is not part of the eye glasses frame having wireless communication module, configured to be attached to different parts of the body. (and configured to submit a substance dosage calculated by the system to be suitable for the treatment of the user in real time)

16. The health monitoring system of claim 3 wherein the audio sensor or vibration data is analyzed for testing at least one of: breath, speech properties, breath pattern volume, air flow, changes in lungs volume.

17. The health monitoring system of claim 3 wherein the humidity sensor data is analyzed for testing at least one of: sweating, conductivity Electrodermal activity, GSR.

18. The health monitoring system of claim 1 wherein the monitoring application further using raw measurement data of an additional wearable device, wherein the wearable device include at least one: smart watch, in-ear device.

19. The health monitoring system of claim 1 further comprising a second Replaceable elastic or semi elastic temple tip, design to be connected to the second end frame of existing eye glasses, said temple tip including at least one optic sensor, CPU, memory, energy source and communication module.

20. The health monitoring system of claim 18 further including energy management module of the two energy sources, based on usage of the sensors at each temple tip.

21. The health monitoring system of claim 18 wherein the monitoring module includes analysis of sweat, body temperature. GRP and heart rate for applying polygraph based algorithm. (need to describe what exactly it does)

22. The health monitoring system of claim 10 where in the AI learning algorithm include a deep learning algorithm configured to for detection and prediction of CVD conditions, wherein the deep learning is based on user profile inputs, user feedback received in real time which are compared with measured health data from the system sensors.

23. The health monitoring system of claim 10 further comprising an actuator for controlling the dosing of the medical substance.

24. The health monitoring system of claim 10 further comprising an elastic band connecting the two temple tips enabling to keep the temple tip close to the skin.

25. The method of claim 1 further comprising the steps of: Receiving blood measured parameter from the patch, checking if blood measured parameters exceeds predefined values, sending an alert to the user.

26. The method of claim 25 applying an AI model using sensor data of blood measured parameters with at least on of BCG, PPG, ECG and PTT to identify patient cardiologic activity, cardiovascular disorder, based client profile and sensor data from the mobile application.
